(19) United States
(12) Patent Application Publication
(10) Pub. No.: US 2019/0049388 A1
(43) Pub. Date: Feb. 14, 2019

(54) FERMENTATION GAS SENSOR SYSTEM

(71) Applicant: BLACK JOULE, LLC, Rapid City, SD (US)

(72) Inventor: Kristophor Ray JENSEN, Rapid City, SD (US)

(73) Assignee: BLACK JOULE, LLC, Rapid City, SD (US)

(21) Appl. No.: 16/101,230

(22) Filed: Aug. 10, 2018

Related U.S. Application Data
(60) Provisional application No. 62/544,390, filed on Aug. 11, 2017.

(51) Int. Cl. G01N 21/78; G01N 33/00; G01N 21/01; G01N 21/63; G01N 21/25; G01N 21/31

(52) U.S. Cl. CPC G01N 21/783; G01N 33/0047; G01N 33/0054; G01N 21/01; G01N 21/63; G01N 33/004; G01N 2021/3129; G01N 21/255; G01N 21/31; G01N 2021/0181; G01N 2033/0068; G01N 2021/0131; G01N 2021/0143; G01N 33/0044

(57) ABSTRACT

A gas sensor system for use in an exhaust gas tube, the system having at least two separate optical sensor assemblies that are separately positioned across the exhaust gas tube from one another, wherein the optical sensor assemblies each comprise a pair of light sources and a pair of light receivers such that light from each of the multiple light sources is received by each of the four light receivers, thereby generating multiple sets of optical measurements.

### RELATED APPLICATION

The present invention claims priority to U.S. Provisional Patent Application Ser. No. 62/544,390 of same title, filed Aug. 11, 2017, the entire disclosure of which is incorporated herein by reference in its entirety for all purposes.

### TECHNICAL FIELD

The present invention relates to gas sensors in general and to alcohol fermentation gas sensors in particular.

### BACKGROUND OF THE INVENTION

Fermentation requires monitoring of several parameters. For industrial applications, it is desirable to monitor a number of different properties of the gasses within the fermenter. Such properties can include determining levels of carbon dioxide, ethyl alcohol, hydrogen sulfide, and water vapor. In addition, it is also desirable to monitor static and dynamic pressures and gas temperature. Depending on the desired product of the fermentation process, measuring methane and acetic acid levels may also desirable. Optionally, the detection of Butanoic Acid, Diacetyl Acid and Propionic acid may also be desired. Therefore, there is need within the fermentation industry for a modular system using customized sensor technology and metadata to accurately measure and provide properties of a wide variety of gasses in a repeatable manner. In contrast, currently available (or traditional) sensors have readings that drift over time resulting in quality control staff making taking multiple manual measurements to acquire and manage the data for maximizing the fermentation process for their specific needs. Lastly, it is also important that any fermentation data collection process does not itself contaminate or physically interfere with the gas it is measuring. As such, when measuring gasses that are passing through pipes or tubes, it is desirable that the gas sensor(s) be as small as possible so as not to disrupt the flow of the gas.

As will be shown, the present sensor system provides user specific measurements of the entire fermentation process by collecting volumes of data from multiple sensors that are used to calculate real-time properties of multiple parameters of the fermenting material (gas, liquid, and solids). These sensed parameters can then be further processed to estimate other values including Specific Gravity, rate of fermentation, and the detection of fermentation problems.

### SUMMARY OF THE INVENTION

The present gas sensor system is comprised of module(s) that are located on the fermenter exhaust gas tube (or other gas tubes). In its various embodiments, the present sensor system is ideally suited for use with any type of fermenter including but not limited to beer, wine, alcohol, ethanol, waste treatment, composting, etc.

In preferred aspects, data from the present sensor system can be stored locally, or on a server or in the Cloud and be manipulated by various algorithms to monitor the fermentation process (or any other process being monitored by the present sensor system). In various embodiments of the present system, some of the data may optionally be processed on a computer system remote from the server. However, it is to be understood that the present system is not limited to requiring external processing.

In preferred aspects, the present sensor system comprises a gas sensor system that may include two separate sensor assemblies, comprising: (a) a first optical sensor assembly comprising: first and second light sources, first and second light receivers; and (b) a second optical sensor assembly comprising: first and second light sources and first and second light receivers. Preferably, the first and second optical sensor assemblies are positioned across from one another within the exhaust gas tube. In optional embodiments, various gas sensor assemblies can be added, and these can also be positioned in the exhaust gas tube.

Additionally, either or both of the optical sensors can comprise a mirror or reflective surface thereon, permitting light to be reflected off of either of the two optical sensors. In addition, a separate mirror (that is not part of a sensor assembly) can optionally be used to bounce light back towards a single optical sensor. In such embodiments, the mirror can be positioned across an exhaust gas tube from the single optical sensor.

In preferred embodiments, the first and second light sources in the first optical sensor assembly direct light to the first and second light receivers in the second optical sensor assembly, and the first and second light sources in the second optical sensor assembly direct light to the first and second light receivers in the first optical sensor assembly. As a result, at least four sets of optical measurements are generated.

Preferably, each of the separate sensor assemblies are mounted on separate printed circuit boards and have their own dedicated microcontroller, power supply and wired or wireless data transmitter configured to transmit the measured data to the Cloud. These sensor assemblies are preferably positioned in an exhaust gas tube such that the two optical sensor assemblies are positioned directly across from one another on the inner sides of the exhaust tube. Optionally, a gas sensor may be positioned mid-way between the two optical sensors on the inner side of the exhaust tube. In optional embodiments, this gas sensor (or sensors) can be used to detect ethanol, O₂, CO₂, H₂S, and humidity.

In various alternate embodiments, the present system may comprise only two optical sensor assemblies (with the optional gas sensor removed). In further alternate embodiments, the present system may comprise additional optical sensor assemblies, preferably added in pairs such that the present system may comprise one, two, four, six, eight, etc. optical sensor assemblies.

The light sources used may be one or more of broadband white light sources, multi-spectral light sources, infra-red sensors, near infra-red sensors, etc. The light receivers used may optionally be multi-spectral sensors, ultraviolet sensors, visible light sensors, infra-red sensors, near infra-red sensors, etc. As such, the present system encompasses any and all wavelengths of light both in its emitters/sources and in it receivers.

Any of the preferred sensor assemblies may also optionally include static or dynamic pressure sensors and temperature sensors.

The present system also includes a method of sensing exhaust gasses, comprising: (a) positioning two or more separate optical sensor assemblies within an exhaust gas tube, wherein the first and second optical sensor assemblies are positioned across from one another within the exhaust gas tube, and wherein the first and second optical sensor assemblies each comprise a pair of light sources and a pair of light receivers such that light from each of the four light sources is received by each of the four light receivers, thereby generating four sets of optical measurements; (b) measuring gas properties with each of the sensor assemblies; and (c) correlating the data received from each of the separate sensor subassemblies by comparing the four sets of optical measurements.

Correlating the data received from each of the separate sensor subassemblies by comparing the four or more sets of optical measurements can comprise averaging the four sets of optical measurements into a single optical measurement. As a result, several simultaneous readings of the same gas property can be taken and compared to one another for accuracy. Specifically, the four or more sets of optical measurements from the sensor assemblies can be transmitted to a database resident on a server or in the Cloud. Cloud based software can then be used to analyze properties of the gas, and optionally determine the identities of various gasses in the exhaust gas tube.

The present system is ideally suited for use within the exhaust gas tube of a fermenter. A further advantage of the present system is that the sensor assemblies are not placed within the fermenter itself. Thus, unlike existing sensors, they do not penetrate the pressurized tank itself.

In further aspects, the present system covers various embodiments of gas sensors positioned on exhaust pipes (where gasses in a vessel exit the vessel). Thus, the present system encompasses optional gas sensors positioned on gas exhaust lines that connect to sealed vessels above the fluid line in a vessel.

### BRIEF DESCRIPTION OF THE DRAWINGS

FIG. 1 is a perspective illustration of three sensor assemblies positioned within the exhaust tube of a fermenter.

FIG. 2 is a top plan view corresponding to FIG. 1 (showing light paths between the two optical sensor assemblies).

FIG. 3A is an illustration of the light paths from the first optical sensor to the second optical sensor.

FIG. 3B is an illustration of the light paths from the second optical sensor to the first optical sensor.

FIG. 3C is an illustration of the light paths when only one optical sensor and one mirror are used.

FIG. 4 is an illustration of an exemplary optical sensor.

FIG. 5 is an illustration of a fermenter showing placement of the present sensing system.

FIG. 6 is a top plan view of four optical sensors positioned in an exhaust gas tube (showing light paths between corresponding pairs of optical sensors).

FIG. 7 is a top plan view of six optical sensors positioned in an exhaust gas tube (showing light paths between corresponding pairs of optical sensors).

### DETAILED DESCRIPTION OF THE DRAWINGS

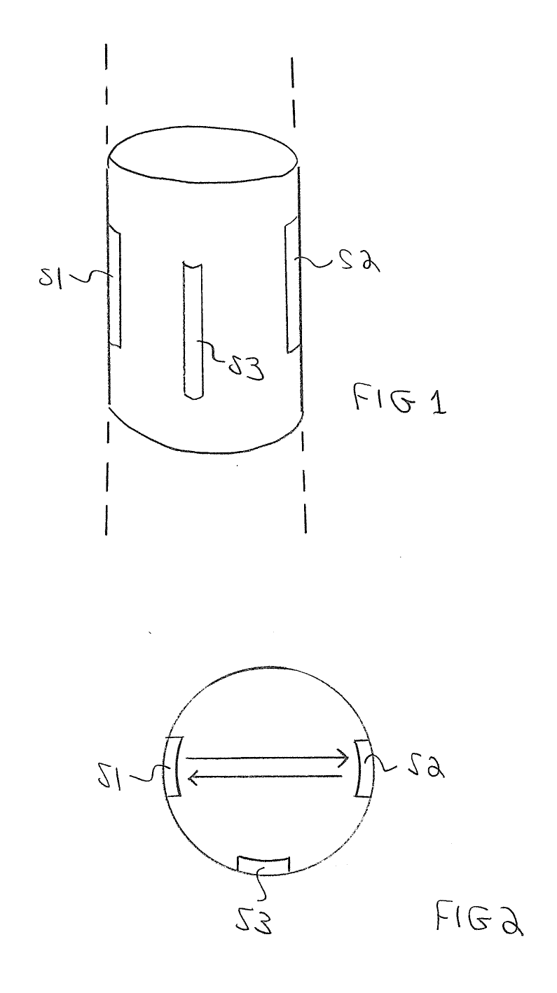

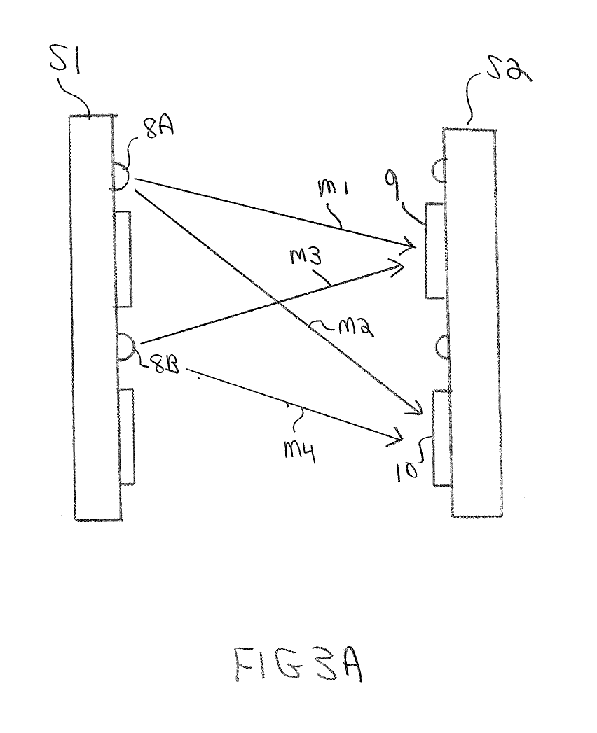

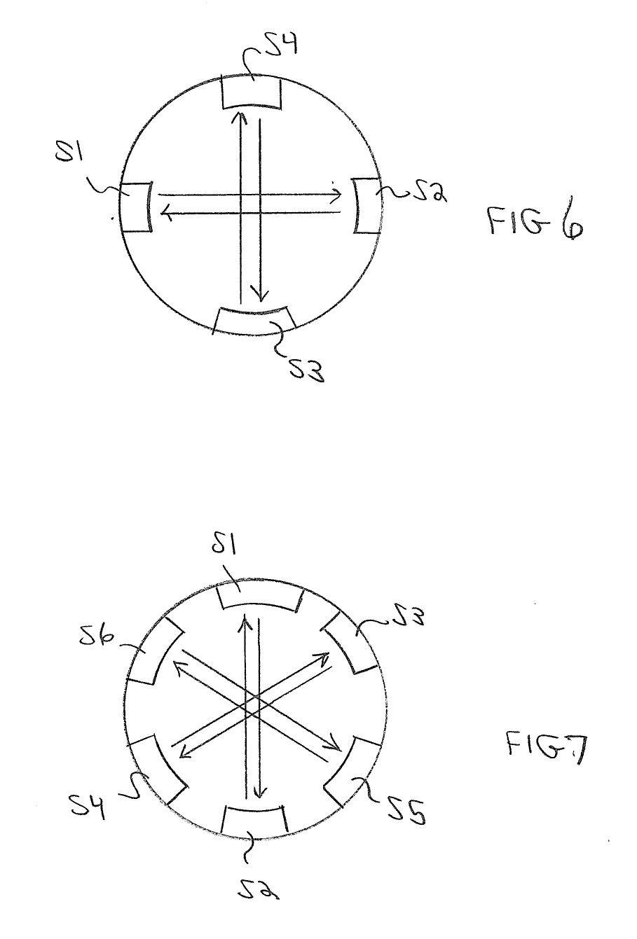

The present gas sensor system preferably comprises separate sensor assemblies, with each sensor assembly being mounted on a separate printed circuit board. As seen in FIGS. 1 and 2, the present system comprises two optical sensors S1 and S2. (An optional gas sensor S3 is also shown). As will be shown later in FIGS. 7 an 8, the presently claimed system also encompasses embodiments with additional pairs of optical sensor assemblies added or removed, and may also optionally include none, one or more than one gas sensor assemblies, all keeping within the scope of the presently claimed system.

Optical sensors S1 and S2 (as seen in FIGS. 1 and 2) can optionally be identical to one another (i.e.: having all of the same components). Optional gas sensor S3 can have some or all of the same components as S1 and S2, but with the optical sources and receivers removed.

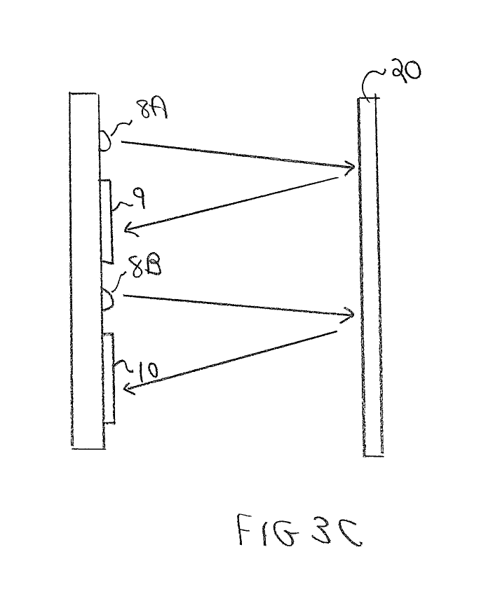

FIG. 4 illustrates an exemplary embodiment of either of optical sensors S1 or S2. Various components of Sensor S1 (or S2) may include a carbon dioxide sensor 100, an ethanol sensor 102, a hydrogen sulfide sensor 104, an oxygen sensor 106, a water vapor sensor 108, a static pressure sensor 110, a dynamic pressure sensor 112, a white light sensor 114, one or more multi-spectral sensors 116 and 118, an ultraviolet sensor(s) 120, an infrared sensor(s) 122. A microcontroller 130, an analog processor 132 and a power supply 134 can also be included in addition with a board connector 140 or a wireless data transmitter 142 configured to transmit measured data to the Cloud. It is to be understood that the present system may have some or all of these components (or additional components), all keeping within the scope of the present invention.

It is also to be understood that sensor assembles that detect different gas properties than those illustrated by the sensors of FIG. 4 are also included within the scope of the present system. The illustration of FIG. 4 is therefore understood to be merely exemplary and are not limiting.

As illustrated in FIGS. 1 and 2, optical sensor assemblies S1 and S2 can be preferably positioned directly across from one another in the exhaust gas tube. This positioning maximizes the amount of gas between the sensors, and also affords advantages in optical sensing since multiple duplicates of the same readings can be taken and analyzed, as follows.

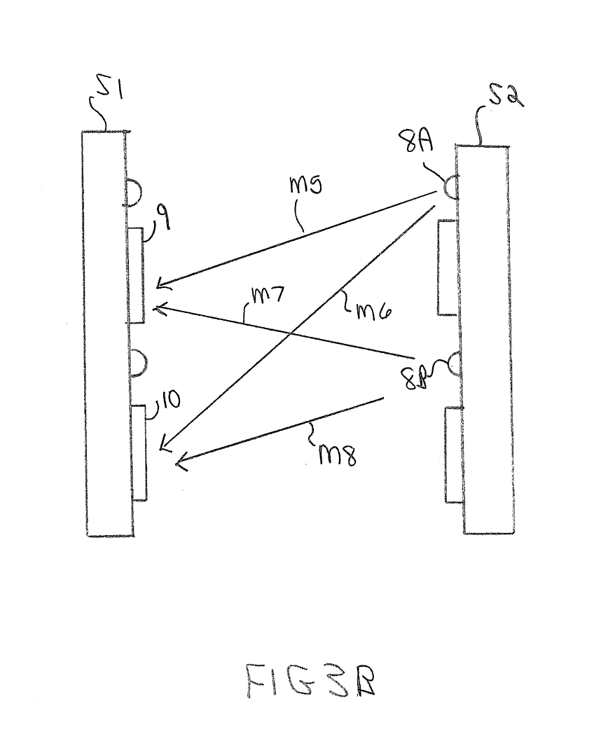

Referring next to FIG. 3A, optical sensor assembly S1 has two broadband white light sources 8A and 8B thereon. Optical sensor assembly S2 has two multi-spectral sensors 9 and 10 thereon. As such, light from the first light source 8A on the first optical sensor S1 assembly is directed to the first light receiver 9 on the second optical sensor assembly S2, thereby generating a first optical measurement M1. Light from the first light source 8A on the first optical sensor S1 assembly is also directed to the second light receiver 10 on the second optical sensor assembly S2, thereby generating a second optical measurement M2.

In addition, light from the second light source 8B on the first optical sensor assembly S1 is directed to the first light receiver 10 on the second optical sensor assembly S2, thereby generating a third optical measurement M3. Lastly, light from the second light source 8B on the first optical sensor S1 assembly is also directed to the second light receiver 10 on the second optical sensor assembly S2, thereby generating a second optical measurement M4.

Next, as seen in FIG. 3B, optical sensor assembly S2 also has two broadband white light sources 8A and 8B thereon. Optical sensor assembly S1 has two multi-spectral sensors 9 and 10 thereon. As such, light from the first light source 8A on the second optical sensor S2 assembly is directed to the first light receiver 9 on the first optical sensor assembly S1, thereby generating a fifth optical measurement M5. In addition, light from the second light source 8B on the second optical sensor assembly S2 is directed to the second light receiver 10 on the first optical sensor assembly S1, thereby generating a sixth optical measurement M6.

In addition, light from the second light source 8B on the second optical sensor assembly S2 is directed to the first light receiver 9 on the first optical sensor assembly S2, thereby generating a seventh optical measurement M7. Lastly, light from the second light source 8B on the second optical sensor S2 assembly is also directed to the second light receiver 10 on the first optical sensor assembly S1, thereby generating an eighth optical measurement M8.

The light paths and resulting data measurements shown in FIG. 3A are preferably taken at the same times as the light paths and resulting data measurements shown in FIG. 3B. Therefore, the first and second light sources in the first optical sensor assembly direct light to the first and second light receivers in the second optical sensor assembly, at the same time that the first and second light sources in the second optical sensor assembly direct light to the first and second light receivers in the first optical sensor assembly. As a result, eight sets of optical measurements are generated at the same time. The advantage of this approach is that the multiple sets of optical measurements can be compared to one another and averaged into a single (highly accurate) optical measurement. Thus, redundancies are provided in the system, and any “drift” in any one sensor will be compensated by the presence of the other sensors/light paths. In other data analysis techniques, a single (highly accurate) optical measurement could be determined by averaging the closest three of the measurements (since an outlying measurement recorded in only one sensor may instead be indicative of sensor malfunction).

FIG. 3C shows an optional embodiment of the present system in which a single sensor S1 and a mirror 20 are positioned on opposite sides of a gas exhaust tube and light from light source 8A is reflected into light receiver 9 and light from light source 8B is reflected into light receiver 10.

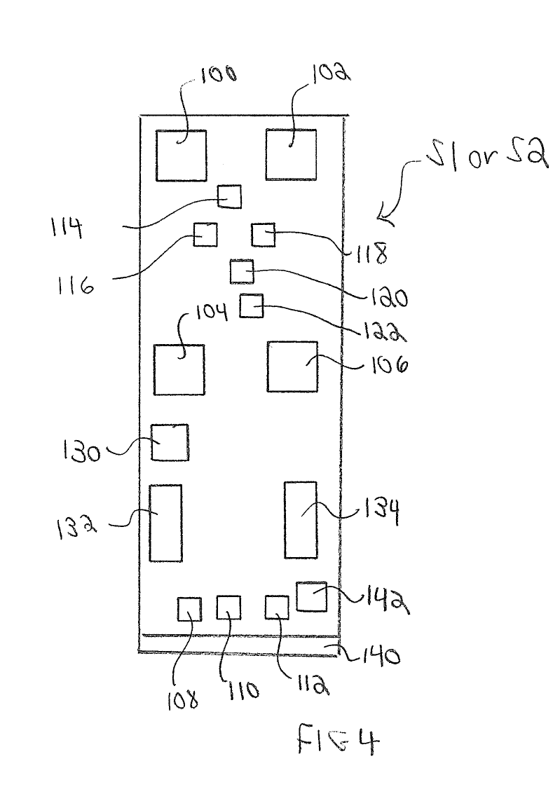

FIG. 5 illustrates placement of the present sensors at location 210, being in an exhaust gas tube 205 connected to a fermentation tank 200. In preferred aspects, location 210 is above tank fluid level 201 to permit sending of the gas leaving the fermentation tank 200.

It is to be understood that in accordance with the present system, additional sensors and light paths may be added (or removed). Rather, the advantage of the present system is that any measurement (from a light source on S1 to a sensor on S2) can itself be replicated (with light passing from a light source on S2 to a sensor on S1).

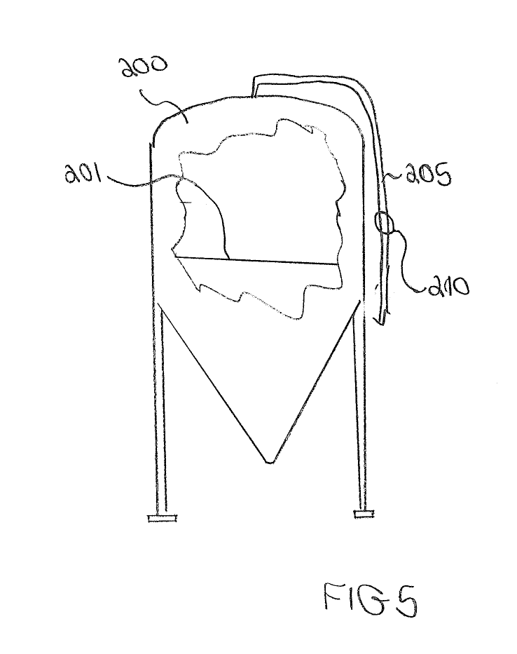

For example, FIG. 6 illustrates four sensor assemblies (S1, S2, S3 and S4). In this illustration, all of sensor assemblies S1, S2, S3 and S4 are optical sensor assemblies. Light passes between the light sources and receivers on sensor assemblies S1 and S2 exactly as described above with respect to FIGS. 1 to 3B. However, in FIG. 6, light also passes between the light sources and receivers on sensor assemblies S3 and S4. Thus, optical sensors S3 and S4 give further data such that eight additional sets of data can be analyzed (similar to the eight sets of data produced between optical sensors S1 and S2 as explained in FIGS. 3A and 3B).

FIG. 7 shows six optical sensor assemblies arranged in pairs. In this embodiment, optical sensor assemblies S1 and S2 work together and optical sensor assemblies S3 and S4 work together as was described above. Similarly, optical sensor assemblies S5 and S6 also work together, generating further sets of data. As can be appreciated, the presently claimed system can include any numbers of pairings of optical sensor assemblies.

Additional sensing components (not shown in FIG. 4) can be placed on one, two or three of sensor assemblies S1, S2 and S3 . . . Sₙ. Such additional sensing components can include: a static pressure sensor; a dynamic pressure sensor; and a temperature sensor. Moreover, further additional sensing components can include: a methane sensor; and an acetic acid sensor. The advantage of the present system is that by including the same sensors on more than on assembly, redundant readings can be taken and analyzed. For example, two or three of assemblies S1, S2 and S3 may all include a temperature sensor (i.e.: thermometer). Therefore, the temperature can be measured by each of the two or three assemblies and then averaged to yield a more accurate temperature result.

It is to be understood that the light sources used by assemblies S1 and S2, etc. may comprise broadband white light sources and sensors, ultraviolet light sources and sensors, infra-red light sources and sensors, near infra-red and sensors, etc. Thus, the present system encompasses any and all wavelengths of light both in its emitters/sources and in it receivers.

Multi-spectral sensors 116 and 118 can operate to detect spectral absorption in the 440 nm to 860 nm range; and ultraviolet sensor 120 can operate to detect spectral absorption in the 220 nm-260 nm and 280 nm ranges. It is to be understood that these ranges are merely exemplary, and that the presently claimed system will work with any preferred spectral range detector operating at any wavelength.

The present system also provides a method of sensing exhaust gasses, by: (a) positioning separate sensor assemblies S1 and S2 within an exhaust gas tube, wherein sensor assemblies S1 and S2 are optical sensor assemblies. The first and second optical sensor assemblies S1 and S2 are positioned across from one another within the exhaust gas tube. Assemblies S1 and S2 each comprise a pair of light sources 8 (including sources 114, 120, 122) and a pair of light receivers 9 such that light from each of the four light sources is received by each of the four light receivers. This generates four sets of optical measurements. Next, gas properties are measured with each of the three separate sensor assemblies S1, S2 and S3 (with S1 and S2 working together). Finally, the data received from each of the three separate sensor subassemblies is correlated by comparing the four sets of optical measurements. This can be done using Cloud based software to analyze properties of the gas, and determine the identities of various gasses in the exhaust gas tube.

FIG. 5 is an illustration of a fermenter showing placement of the present sensing system within the exhaust gas tube of the fermenter. It is to be understood that all of the dimensions illustrated in FIG. 5 are merely exemplary, and that the present system is not limited to use just with this particular fermenter. In fact, the present system can be used in any gas tube, pipe or vessel, all keeping within the scope of the present invention. The present system also includes any optical sensor arrangement wherein the sensor assemblies are placed on an exhaust tube from a fermenter or other vessel) at a location 210 above the liquid line 201 in the vessel 200.

In still further embodiments, the present system comprises various numbers of gas (i.e.: non-optical) sensor assemblies including carbon dioxide sensors, ethyl alcohol sensors, hydrogen sulfide sensors, oxygen sensors, water vapor sensors, and may also optionally employ sensor systems that heat a gas and then detect the ions from the heated gas, or membrane sensors where the resistance of the membrane changes in the presence of an ionized gas.

What is claimed is:

1. A gas sensor system having three separate sensor assemblies, comprising:
a first optical sensor assembly; and
a second optical sensor assembly;
wherein each of the first and second sensor assemblies are configured to be separately positioned within an exhaust gas tube such that the first and second optical sensor assemblies are positioned across from one another within the exhaust gas tube.

2. The gas sensor system of claim 1, wherein:
the first and second light sources in the first optical sensor assembly direct light to the first and second light receivers in the second optical sensor assembly, and
the first and second light sources in the second optical sensor assembly direct light to the first and second light receivers in the first optical sensor assembly.

3. The gas sensor system of claim 2, wherein:
the first and second optical sensor assemblies generate four sets of optical measurements.

4. The gas sensor system of claim 1, wherein the first and second optical sensor assemblies are mounted on separate printed circuit boards.

5. The gas sensor system of claim 1, wherein the first and second optical sensor assemblies further comprise:
a static pressure sensor;
a dynamic pressure sensor; and
a temperature sensor.

6. The gas sensor system of claim 1, wherein the first and second optical sensor assemblies further comprise:
a methane sensor; and
an acetic acid sensor.

7. The gas sensor system of claim 1, wherein each of the first and second sensor assemblies further comprise:
a microcontroller;
a power supply; and
a wired or wireless data transmitter configured to transmit measured data to the Cloud.

8. The gas sensor system of claim 1, wherein a third sensor assembly is positioned intermediate the first and second sensor assemblies on the inner side of the exhaust gas tube.

9. The gas sensor system of claim 1, wherein the first and second light sources in each optical assembly comprise at least one of:
a broadband white light source;
an ultraviolet light source; and
an infra-red light source.

10. The gas sensor system of claim 1, further comprising a third light source, wherein the first light source is a broadband white light source, the second light source is an ultraviolet light source, and the third light source is an infra-red light source.

11. The gas sensor system of claim 1, wherein the first and second light receivers in each optical assembly comprise a pair of multi-spectral sensors, or ultraviolet sensors.

12. A gas sensor system having three separate sensor assemblies, comprising:
a first optical sensor assembly, the first optical sensor assembly comprising:
first and second light sources,
first and second light receivers, and
at least one sensor;
a second optical sensor assembly, the second optical sensor assembly comprising:
first and second light sources,
first and second light receivers, and
at least one sensor;
wherein each of the first and second sensor assemblies are configured to be separately positioned within an exhaust gas tube such that the first and second optical sensor assemblies are positioned across from one another within the exhaust gas tube, and
wherein:
(i) light from the first light source on the first optical sensor assembly is directed to the first light receiver on the second optical sensor assembly, thereby generating a first optical measurement,
(ii) light from the first light source on the first optical sensor assembly is directed to the second light receiver on the second optical sensor assembly, thereby generating a second optical measurement,
(iii) light from the first light source on the second optical sensor assembly is directed to the first light receiver on the first optical sensor assembly, thereby generating a third optical measurement, and
(iv) light from the second light source on the second optical sensor assembly is directed to the second light receiver on the first optical sensor assembly, thereby generating a fourth optical measurement.

13. The gas sensor system of claim 12, wherein each of the first and second sensor assemblies further comprise:
a microcontroller;
a power supply; and
a wired or wireless data transmitter configured to transmit measured data to the Cloud.

14. The gas sensor system of claim 12, wherein the two optical sensor assemblies are positioned across from one another on the inner sides of the exhaust gas tube.

15. A method of sensing exhaust gasses, comprising:
positioning first and second sensor assemblies within an exhaust gas tube, wherein the first and second sensor assemblies are optical sensor assemblies,
wherein the first and second optical sensor assemblies are positioned across from one another within the exhaust gas tube, and
wherein the first and second optical sensor assemblies each comprise a pair of light sources and a pair of light receivers such that light from each of the multiple light sources is received by each of the four light receivers, thereby generating multiple sets of optical measurements;
measuring gas properties with each of the first and second separate sensor assemblies; and
correlating the data received from each of the first and second sensor subassemblies by comparing the four sets of optical measurements.

16. The method of claim 15, wherein correlating the data received from each of the first and second sensor subassemblies by comparing the multiple sets of optical measurements comprises averaging the four sets of optical measurements into a single optical measurement.

17. The method of claim 15, wherein measuring gas properties with each of the first and second separate sensor assemblies comprises measuring any one of:
carbon dioxide level,
ethyl alcohol level,
hydrogen sulfide level,
oxygen level,
water vapor level,
static pressure,
dynamic pressure, and
temperature.

18. The method of claim 15, further comprising:
transmitting the multiple sets of optical measurements from the sensor assemblies to the Cloud.

19. The method of claim 18, further comprising:
using Cloud based software to analyze properties of the gas.

20. The system of claim 15, wherein the sensors are positioned above the fluid line in the vessel.
